(19) United States
(12) Patent Application Publication
(10) Pub. No.: US 2004/0137464 A1
(43) Pub. Date: Jul. 15, 2004

(54) Parallel analysis of single nucleotide polymorphisms

(76) Inventors: Hans-Peter Beck, Allschwil (CH); Ingrid Felger, Allschwil (CH); Thomas Moritz, Aesch (CH); Richard Bischof, Basel (CH)

Correspondence Address: GRIFFIN & SZIPL, PC, SUITE PH-1, 2300 NINTH STREET, SOUTH, ARLINGTON, VA, 22204, US

(21) Appl. No.: 10/623,715

(22) Filed: Jul. 22, 2003

Related U.S. Application Data
(60) Provisional application No. 60/397,556, filed on Jul. 22, 2002.

(51) Int. Cl. C12Q 1/68; C12P 19/34

(52) U.S. Cl. 435/6; 435/91.2

(57) ABSTRACT

The present invention relates to a method and apparatus for the analysis of single nucleotide polymorphisms (SNP) in a target DNA. The present invention is based on primer extension on preferably amplified gene fragments using labeled, preferably fluorochrome labeled, dideoxynucleotides, thus allowing extension of only one nucleotide. Subsequently, extended primers are hybridized to immobilized antisense oligonucleotides. SNP analysis is then performed using a suitable detection method, preferably a multi-laser scanner, identifying the respective nucleotides by the label, preferably the fluorochrome label, and followed by analysis of the hybridization pattern.

### CROSS-REFERENCE TO RELATED APPLICATIONS

This application claims benefit under 35 U.S.C. 119(e) of U.S. Provisional Application 60/397,556, filed Jul. 22, 2002, the entire disclosure of which is hereby incorporated by reference.

### BACKGROUND OF THE INVENTION

1. Field of the Invention

The present invention pertains to a measuring or testing process involving nucleic acids. More specifically, the invention is directed to a method and apparatus for the analysis of single nucleotide polymorphisms (SNPs) in a target DNA.

2. Background Art

Single nucleotide polymorphisms (SNPs) provide, by their multiple variations, information on, e.g., disease susceptibility, pharmacogenetics, and drug susceptibility.

In the case of malaria it has been known that drug resistance is conferred by or at least associated with an accumulation of SNPs either in genes coding for target enzymes of a drug or, for example, for transporters associated with drug influx and efflux.

Hitherto known monitoring methods for SNPs are performed mainly by polymerase chain reaction (PCR) amplification or by sequence-specific oligonucleotide (SSPO) analysis. All known techniques involve many processing steps, fine tuning of conditions, expensive enzymes, and are not sufficiently robust for large-scale applications. For example, genotyping for drug resistance by analyzing one SNP in pfmdr, one in pfcrt and four SNPs each in pfdhps and pfdhfr, requires six PCR reactions and seventeen subsequent restriction digests, plus electrophoresis gel analysis.

To use the intrinsic information of SNPs for diagnosis or for medical and molecular epidemiology studies, a high throughput technology to analyze SNPs is needed.

### SUMMARY OF THE INVENTION

It is a general object of the invention to permit the analysis of single nucleotide polymorphisms (SNPs) in a target DNA. In one embodiment of the invention, the method comprises the following steps:

(a) conducting a primer extension reaction on the target DNA using labeled dideoxynucleotides and an oligonucleotide primer having a sequence such that upon hybridization to the target DNA, the 3′ end of the oligonucleotide primer terminates at the last nucleotide before a particular single nucleotide polymorphism. This primer extension reaction produces an extended primer with the 3′ end having a labeled dideoxynucleotide corresponding to the single nucleotide polymorphism in the target DNA;

(b) hybridizing the extended primer to one or more oligonucleotides immobilized on a solid support in the form of an immobilization pattern, whereby a hybridization pattern is produced; and

(c) detecting the presence or absence of hybridized extended primers in the hybridization pattern.

As used in this description and in the appended claims, “dideoxynucleotides” (“ddNTPs”) also includes acycloterminators and any other nucleotide or non-nucleotide terminators that could be used to terminate a polymerase reaction.

According to one preferred embodiment of the invention, before the primer extension reaction, the target DNA harboring one or more SNPs, also called the template DNA, is amplified by PCR using sequence specific primers to produce additional target DNA. The additional target DNA is then treated with an alkaline phosphatase such as shrimp alkaline phosphatase (SAP) and used in place of or together with the target DNA in the primer extension reaction. Taq polymerase may preferably be used for target amplification.

In a further preferred embodiment, the PCR is a multiplex PCR.

According to a still further embodiment, the primer extension reaction is a multiplex primer extension reaction.

According to yet a further preferred embodiment, the initial step consists of first identifying a single nucleotide polymorphism of interest in the target DNA. As used in this description and in the appended claims, “identifying” can mean looking up or discovering experimentally.

According to a still further embodiment, one or more of the SNPs is associated with drug resistance.

According to yet a further embodiment, one or more of the SNPs are located in genes coding for target enzymes of a drug or for transporters associated with drug influx or efflux.

As used in this description and in the appended claims, “associated with” means known or suspected to be directly or indirectly involved in, or known or suspected to be material to.

According to a still further embodiment, the oligonucleotide primers have a length between 20 and 40 base pairs.

According to yet a further embodiment, the target DNA is from a microorganism.

According to a still further embodiment, the target DNA is from a pathogen.

According to yet a further embodiment, the target DNA is from the taxon Apicomplexa.

According to a still further embodiment, the target DNA is from the genus Plasmodium.

According to yet a further embodiment, the target DNA is from the species Plasmodium falciparum.

According to a still further embodiment, one or more of the SNPs are located in one or more Plasmodium falciparum genes selected from the group consisting of pfmdr-1, pfcrt, pfdhfr, pfdhps, pftctp, and the Cytochrome-B gene.

According to yet a further embodiment, the ddNTPs are fluorochrome labeled, and preferably each of a plurality of species of ddNTP is labeled with a different fluorochrome.

The number of SNPs simultaneously analyzed in one primer extension involving a single target DNA reaction can vary and said number depends on the specific case. It is therefore possible that in a specific case about 15 SNPs can be analyzed and in another specific case about 75 SNPs can be analyzed, i.e. there is no general absolute upper limit to the number of SNPs that can be analyzed by the method of the present invention.

According to a still further embodiment, at least about 2 SNPs of the target DNA are analyzed in one said primer extension reaction, preferably at least about 10 SNPs, more preferably at least about 25 SNPs, and even more preferable at least about 50 SNPs.

According to yet a further embodiment, the immobilized oligonucleotides are immobilized on a microarray.

According to a still further embodiment, the immobilized oligonucleotides are immobilized on an aldehyde slide using a C6 amino linker.

According to yet a further embodiment, a SNP involved in drug resistance in malaria is identified and monitored.

According to a still further embodiment, a SNP of interest for diagnostic or pharmacogenetic analysis is identified and monitored.

According to yet a further embodiment, the PCR is done in situ.

According to a still further embodiment, an apparatus performs the steps of the above method.

Further objects, features and advantages of the present invention will become apparent from the Detailed Description of Preferred Embodiments, which follows, when considered together with the attached Figures.

### BRIEF DESCRIPTION OF THE DRAWINGS

FIG. 1 illustrates an embodiment of an application system for analysis of SNPs in accordance with the present invention.

FIG. 2 is an overhead view of the multiprocedure station.

FIG. 3 is a side view of the multiprocedure station.

### DETAILED DESCRIPTION OF THE PREFERRED EMBODIMENTS

The present invention provides a robust, inexpensive and fast genotyping system for single nucleotide polymorphisms, also known as point mutations.

The method of the present invention is based on liquid primer extension of preferably amplified gene fragments using labeled (e.g. fluorochrome labeled), dideoxynucleotides, thus allowing extension of only one nucleotide. Subsequently, extended primers are hybridized to immobilized antisense oligonucleotides. SNP analysis is then performed using a suitable detection method, for example a multi-laser scanner to identify the respective nucleotides by their labels. Preferrably the detecting step can be carried out by detecting the fluorochomic quality or color of the hybridized extended primers.

The method of the present invention is a suitable genotyping system for molecular drug resistance monitoring. For example, the method allows strain identification of infectious species, such as, e.g., unicellular parasites from the taxon Apicomplexa. It also allows the identification and analysis of polymorphic vaccine antigens for monitoring vaccine efficacy and the analysis of break-through parasitemia in ongoing vaccine trials.

The present invention also permits the detection and analysis of SNPs for human susceptibility factors such as ICAM1 promoter SNPs and TNFα promoter SNPs in the analysis. The method and apparatus would enhance our understanding of host and parasite polymorphisms dramatically.

The invention can also be used in other fields of medicine, e.g., diagnosis and pharmacogenetics.

In a preferred embodiment of the present invention, a low density array for all known SNP sites within the P. falciparum genes implicated in resistance against antimalarial drugs is generated and used in the hybridization step of the present method. Preferably a fluid system is used (as part of an immunofluorescence assay (IFA)) for hybridization in order to be able to hybridize multiple samples on one microarray. The inclusion of a microarray in the present invention has several advantages: flexibility to various surfaces and arrays, adaptability, readily available equipment in many laboratories (not bound to a particular instrument), low cost, and the large potential of standardization of technique and protocols.

In a preferred embodiment of the present invention, both the optional PCR amplification of gene fragments harboring SNPs and the subsequent primer extension reaction are multiplex reactions, thereby reducing processing steps and costs. The use of a multiplex primer extension reaction allows the scanning for a large number of SNPs simultaneously in one sample. For the analysis of SNPs in close proximity (<30 base pairs apart), it is preferred to design one primer on the (+) strand and the other primer on the (−) strand. Furthermore, it is possible to semi-quantify the primer extension reactions. This aspect is of great importance in Plasmodium falciparum malaria, where infections are often caused by a multitude of different strains.

In a preferred embodiment of the invention, the PCR reaction occurs, preferably in a microtiter plate, using a commercial PCR machine. The PCR products are purified, preferably using a microtiter manifold, before being used as template DNA in the primer extension reaction.

In another preferred embodiment, the primer extension reaction is performed in a microtiter plate using a commercial PCR machine. Using a microtiter plate for primer extension gives higher sensitivity than methods performing primer extension on a chip or microarray.

Persons skilled in the art know general molecular biology methods needed to practice the present invention. They are described in, for example, Molecular Cloning: A Laboratory Manual, Joseph Sambrook et al., Cold Spring Harbor Laboratory Press, 2000. PCR methods are known to the person skilled in the art and are described in, for example, PCR Primer: A Laboratory Manual, Carl W. Dieffenbach and Gabriela S. Dveksler, Eds., 1998.

For PCR target amplification, Taq polymerase is preferred, although any heat-stable DNA polymerase can be used. For primer extension, Sequenase (TM) is preferred, although another DNA polymerase can be used. Primer extension preferably occurs at 72 degrees Celsius.

Suitable labels for the ddNTPs used in this invention are, for example, the Cy4 and Cy5 fluorochromes. The use of two or more fluorochromes facilitates discrimination between the SNPs in question. Preferably each of the four species of ddNTPs is labeled with a different fluorochrome e.g. including with R6G and a Cy7 derivative. The four fluorochrome-labeled ddNTPs preferably show excitation spectra for suitable for standard dual laser scanners, distinguishable emission spectra, and kinetics suitable for polymerases.

Referring now to the drawings, the structural details and the operation of an apparatus embodiment of the present invention is illustrated.

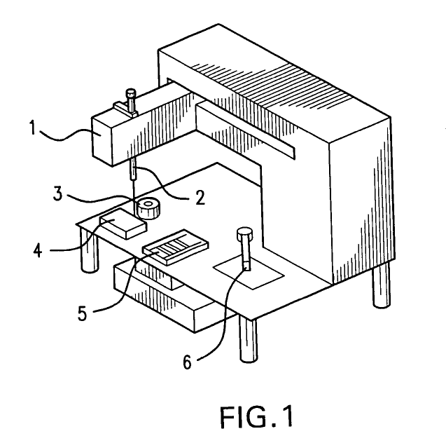

Referring now to FIG. 1, a view of the application system is shown. The application system consists of a xyz-robot arm 1, an active dispenser 2, an active washing-station 3, a primer-station 4, and a multiprocedure station 5 with a cover plate 6. The arrangement of the xyz-robot arm 1, the active dispenser 2, and the active washing-station 3 is also referred to as an automated pipetting robot. The application system performs automatic pipetting and spotting procedures and can operate in a high humidity environment. The pickup unit of the dispenser 2 can be cleaned actively. The primer-station 4 holds a microtiter plate or other source of one or more completed primer extension reactions. The multiprocedure-station 5 can hold one or more microarrays such as 3×12 well glass microarrays. The multiprocedure-station 5 is used for hybridization in a relative humidity of up to 100% and, in certain embodiments of the invention, for washing and drying of the microarrays following hybridization.

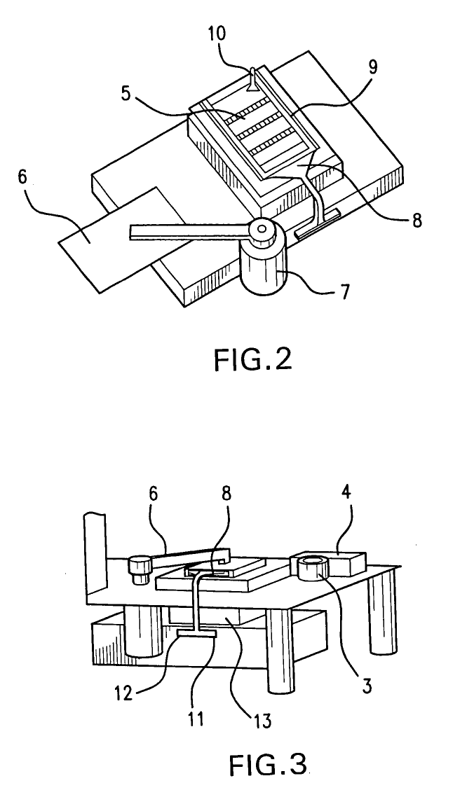

Referring now to FIG. 2, an overhead view of the multiprocedure station 5 is shown. The microarrays are situated in the interior the multiprocedure station 5. The cover plate 6 of the multiprocedure station 5 can be closed with a mechanical device 7 to seal the interior of the multiprocedure station. A distribution unit 8 serves to fill the channel 9 with water, washing solution, or other solution. A discharge port 10 permits drainage of fluid or venting of gas from the interior of the multiprocedure station.

Referring now to FIG. 3, a side view of the multiprocedure station 5 is shown. The distribution unit 8 is connected to an air tube 12 and a wash tube 11. Following the hybridization, the wash procedure is started. The discharge tube 10 is opened and washing solution is supplied via the distribution unit 8 and wash tube 11. The wash tube 11 may also supply water or other solutions. Continuous washing may be performed. The distribution unit 8 connected to the wash tube 11 is also referred to as a washing assembly. After the washing step, the microarrays may be dried by supplying air or another gas via the distribution unit 8 and air tube 12. The distribution unit 8 connected to the air tube 12 is also referred to as a drying assembly. One or more heating or cooling units 13, preferably a Peltier device, serve to heat or cool the microarrays to facilitate the hybridization and washing. A single device, e.g. a Peltier device, may accomplish both the heating and cooling. The temperature of the device (and thus the microarrays) is programmable and can be set at any point between room temperature and 100 degrees Celsius. A series of such temperature steps can be programmed, each with a duration of 30 seconds or longer.

The following first example further explains use of the invention:

A microtiter plate with one or more completed primer extension reactions is placed in the primer-station 4, and one or more 3×12 well glass microarrays containing immobilized oligonucleotides are placed in the multiprocedure station 5. The dispenser 2 picks up between 5 and 50 microliters of the primer extension reaction mixture from a designated well of the microtiter plate and transfers the mixture to a designated well of a microarray plate. The dispenser 2 is washed in the washing-station 3. The dispenser continues to transfer primer extension reaction mixture(s) to other wells of the microarray plates as programmed, with a wash step after each transfer.

Then cover plate 6 of the multiprocedure station 5 is closed. The interior of the multiprocedure station 5 is humidified with water delivered via the wash tube 11 and distribution unit 8. The interior of the multiprocedure station 5 is heated to 95 degrees Celsius for 2 to 5 minutes using the Peltier elements. Then the interior of the multiprocedure station 5 is cooled and kept at 42 degrees Celsius for 2 hours. Next the discharge tube 10 is opened and washing solution is supplied via the distribution unit 8 and wash tube 11 as programmed. After the washing is complete, the interior of the multiprocedure station 5 is drained and the microarrays are air dried and ready for scanning.

The following second example further explains carrying out the method of the present invention:

First, DNA is prepared using standard methods. Then the target gene(s) are amplified using a commercial PCR machine and Taq polymerase or other heat-stable polymerase. Next the PCR products are purified in preparation for the primer extension reaction.

Oligonucleotide primers of approximately 40 base pairs in length corresponding to the (+) strand sequence of the coding genes implicated in resistance are designed. The primers are targeted to sequences on the (−) strand, placing them with their 3′ end terminating at the last nucleotide before the SNP site. All primers are designed to hybridize at an identical T_{M} value by reducing or increasing their length appropriately. For the analysis of SNPs in close proximity (<30 base pairs apart), one primer is designed for the (+) strand and the other for the (−) strand. Amplification will not occur since primer extension utilizes only ddNTPs (or similar equivalent nucleotides or non-nucleotides), which will not allow extended synthesis of DNA. Otherwise standard conditions for primer extension are used, typically using Sequenase (TM) polymerase.

After primer extension, the reaction is heat denatured and the single stranded extended primers are hybridized onto a microarray spotted with antisense oligonucleotides. The array is produced on an available micro-arrayer in triplicate serving as control. All oligonucleotides are synthesized corresponding to the complementary sequence and to the same length as the primers in the primer extension step, without the extended nucleotides. The oligonucleotides are modified with a C6 amino link and are covalently bound onto aldehyde slides. Standard hybridization protocols are used for hybridization. In order to test spotting efficiency, and as a reference in quantification, pre-labeled oligonucleotides are also arrayed.

The hybridized microarray is washed and read in a commercially available laser scanner. The emission pattern will allow determination of the species of the extended base and the signal ratio will allow quantification of the abundance of each nucleotide. The latter ability is of great importance in Plasmodium falciparum malaria, where infections are often caused by a multitude of different strains.

While the present invention has been described with reference to certain illustrative embodiments, one of ordinary skill in the art will recognize that additions, deletions, substitutions and improvements can be made while remaining within the scope and spirit of the invention as defined by the appended claims.

What is claimed is:

1. A method for detecting a single nucleotide polymorphism in a target DNA, comprising the steps of:
(a) conducting a primer extension reaction with components including (1) the target DNA, (2) labeled dideoxynucleotides, and (3) an oligonucleotide primer having a sequence hybridizable to the target DNA, so that a 3′ end of the oligonucleotide primer terminates at a last nucleotide before a single nucleotide polymorphism, whereby an extended primer is produced including a 3′ end having a labeled dideoxynucleotide corresponding to the single nucleotide polymorphism in the target DNA;
(b) hybridizing the extended primer to one or more oligonucleotides immobilized on a solid support in the form of an immobilization pattern, whereby a hybridization pattern is produced; and
(c) detecting the presence or absence of hybridized extended primer in the hybridization pattern.

2. The method of claim 1, wherein said primer extension reaction is a multiplex primer extension reaction.

3. The method of claim 1, further including the step, before step (a), of identifying a single nucleotide polymorphism of interest in the target DNA.

4. The method of claim 3, wherein one or more of said single nucleotide polymorphisms are associated with drug resistance.

5. The method of claim 1, wherein one or more of said single nucleotide polymorphisms are located in genes coding for target enzymes of a drug or for transporters associated with drug influx or efflux.

6. The method of claim 1, wherein said oligonucleotide primer has a length between 20 and 40 base pairs.

7. The method of claim 1, further comprising the steps, before step (a), of: amplifying the target DNA using sequence-specific primers in a polymerase chain reaction, whereby a product is produced comprising the original target DNA and additional target DNA; and treating the additional target DNA with alkaline phosphatase.

8. The method of claim 7, wherein said polymerase chain reaction is a multiplex polymerase chain reaction.

9. The method of claim 7, wherein the polymerase chain reaction is an in situ polymerase chain reaction.

10. The method of claim 1, wherein said target DNA is from a microorganism.

11. The method of claim 10, wherein said microorganism is a pathogen.

12. The method of claim 11, wherein said pathogen is of a taxon Apicomplexa.

13. The method of claim 12, wherein said pathogen is of the genus Plasmodium.

14. The method of claim 13, wherein said pathogen is of the species Plasmodium falciparum.

15. The method of claim 14, wherein said single nucleotide polymorphism is located in a Plasmodium falciparum gene selected from the group consisting of pfmdr-1, pfcrt, pfdhfr, pfdhps, pftctp, and the Cytochrome-B gene.

16. The methods of claim 1, wherein said dideoxynucleotides are fluorochrome labeled.

17. The method of claim 16, wherein said dideoxynucleotides comprise a plurality of species, each species being labeled with a different fluorochrome.

18. The method of claim 17, wherein said detecting step comprises detecting hybridized extended primers with a multi-laser scanner.

19. The method of claim 1, wherein said detecting step includes detecting the presence or absence of at least about 2 single nucleotide polymorphisms of the target DNA.

20. The method of claim 1, wherein said detecting step includes detecting the presence or absence of at least about 10 single nucleotide polymorphisms of the target DNA.

21. The method of claim 1, wherein said detecting step includes detecting the presence or absence of at least about 25 single nucleotide polymorphisms of the target DNA.

22. The method of claim 1, wherein said detecting step includes detecting the presence or absence of at least about 50 single nucleotide polymorphisms of the target DNA.

23. The method of claim 1, wherein said immobilized oligonucleotides are immobilized in a microarray.

24. The method of claim 23, wherein said microarray consists of an aldehyde slide and said immobilized oligonucleotides are bound to the aldehyde slide with a C6 amino linker.

25. The method of claim 1, wherein said detecting step comprises detecting a fluorochomic quality or color of said hybridized extended primer.

26. A method for drug resistance testing in malaria, comprising the steps of:
(a) identifying a single nucleotide polymorphism related to drug resistance in malaria;
(b) conducting a primer extension reaction with components including (1) the target DNA, (2) labeled dideoxynucleotides, and (3) an oligonucleotide primer having a sequence hybridizable to the target DNA, so that a 3′ end of the oligonucleotide primer terminates at a last nucleotide before a single nucleotide polymorphism, whereby an extended primer is produced including a 3′ end having a labeled dideoxynucleotide corresponding to the single nucleotide polymorphism in the target DNA;
(c) hybridizing the extended primer to one or more oligonucleotides immobilized on a solid support in the form of an immobilization pattern, whereby a hybridization pattern is produced; and
(d) detecting the presence or absence of hybridized extended primer in the hybridization pattern.

27. A method for diagnostic or pharmacogenetic analysis of single nucleotide polymorphisms in a target DNA, comprising the steps of:
(a) identifying a single nucleotide polymorphism of interest for diagnostic or pharmacogenetic analysis;
(b) conducting a primer extension reaction with components including (1) the target DNA, (2) labeled dideoxynucleotides, and (3) an oligonucleotide primer having a sequence hybridizable to the target DNA, so that a 3′ end of the oligonucleotide primer terminates at a last nucleotide before a single nucleotide polymorphism, whereby an extended primer is produced including a 3′ end having a labeled dideoxynucleotide corresponding to the single nucleotide polymorphism in the target DNA;
(c) hybridizing the extended primer to one or more oligonucleotides immobilized on a solid support in the form of an immobilization pattern, whereby a hybridization pattern is produced; and
(d) detecting the presence or absence of hybridized extended primer in the hybridization pattern.

28. An apparatus for analysis of single nucleotides polymorphisms in target DNA, comprising:
a multiprocedure station having a sealable interior able to hold one or more microarrays,
a source of one or more oligonucleotide primers to be added the to microarrays, the primers having labeled 3′ ends corresponding to one or more single nucleotide polymorphisms, and
a heating or cooling unit arranged to heat and/or cool the microarrays.

29. The apparatus of claim 28, further comprising:
a washing assembly arranged to wash the microarrays, and
a drying assembly arranged to dry the microarrays.

30. The apparatus of claim 28, further comprising:
an automated pipetting robot including a xyz-robot arm, an active dispenser, and a wash station arranged to wash the active dispenser, the robot arranged to transfer said primers to said microarrays.
